(19) United States
(12) Patent Application Publication
(10) Pub. No.: US 2023/0119355 A1
(43) Pub. Date: Apr. 20, 2023

(54) PHARMACEUTICAL COMPOSITIONS OF A KINASE INHIBITOR

(71) Applicant: AURIGENE DISCOVERY TECHNOLOGIES LIMITED, Bangalore (IN)

(72) Inventors: Frederic DARGELAS, Espoo (FI); Piritta KOISTINEN, Turku (FI); Laura LEIMU, Naantali (FI); Laura MATILAINEN, Kauniainen (FI); Henri METSALA, Espoo (FI); Anna SHEVCHENKO, Horsholm (DK)

(73) Assignee: AURIGENE DISCOVERY TECHNOLOGIES LIMITED, Bangalore (IN)

(21) Appl. No.: 17/908,521

(22) PCT Filed: Mar. 5, 2021
(86) PCT No.: PCT/FI2021/050162

§ 371 Date: Aug. 31, 2022

(30) Foreign Application Priority Data
Mar. 5, 2020 (FI) 20205233

(51) Int. Cl. A61K 31/4184; A61K 9/16; A61K 47/32

(52) U.S. Cl. CPC A61K 31/4184; A61K 9/1635; A61K 47/32

(57) ABSTRACT

The present invention relates to pharmaceutical compositions comprising hydrochloride salt of N-(2′,4′-difluoro-5-(5-(1-methyl-1H-pyrazol-4-yl)-1H-benzo[d]imidazol-1-yl)-[1,1′-biphenyl]-3-yl)cyclopropanesulfonamide (I) as an active ingredient and copovidone as an excipient. Compound (I) is a selective inhibitor of FGFR/VEGFR kinase families and is useful in the treatment of cancer.

### TECHNICAL FIELD

The present invention relates to pharmaceutical compositions comprising hydrochloride salt of N-(2′,4′-difluoro-5-(5-(1-methyl-1H-pyrazol-4-yl)-1H-benzo[d]imidazol-1-yl)-[1,1′-biphenyl]-3-yl)cyclopropanesulfonamide (I) as an active ingredient.

### BACKGROUND OF THE INVENTION

The compound N-(2′,4′-difluoro-5-(5-(1-methyl-1H-pyrazol-4-yl)-1H-benzo[d]-imidazol-1-yl)-[1,1′-biphenyl]-3-yl)cyclopropanesulfonamide of formula (I) and derivatives thereof have been disclosed in WO 2013/053983. Compound of formula (I) is a selective inhibitor of FGFR/VEGFR kinase families and is useful in the treatment of various cancers, particularly those in which abnormal FGFR signalling has been reported, such as multiple myeloma, gastric cancer, endometrial cancer, prostate cancer, breast cancer, cholangiocarcinoma and uroepithelial carcinoma.

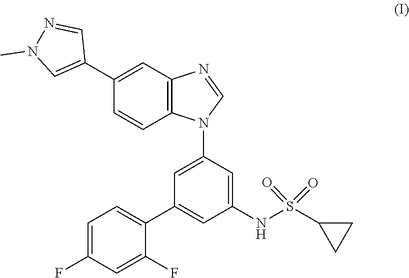 [SMILES: Cn1cc(-c2ccc3c(c2)ncn3-c2cc(NS(=O)(=O)C3CC3)cc(-c3ccc(F)cc3F)c2)cn1]

Compound (I) is practically insoluble in water at physiological pH range and has very low bioavailability after oral administration. It is also poor salt former and appears to be neutral within the physiological pH range. Hydrochloride salt forms of compound (I), for example crystalline form 8, have been found to be suitable for use in the manufacture of stable pharmaceutical products which exhibit enhanced water solubility and improved bioavailability after oral administration. However, there is still a need to improve oral bioavailability in order to reach higher exposure of the active ingredient at the targeted sites.

### SUMMARY OF THE INVENTION

It has now been found that oral bioavailability of hydrochloride salt of compound (I) can be substantially improved if administered in a pharmaceutical composition comprising copovidone (PVP/VA) as an excipient.

Thus, in one aspect, the present invention provides a pharmaceutical composition comprising hydrochloride salt of N-(2′,4′-difluoro-5-(5-(1-methyl-1H-pyrazol-4-yl)-1H-benzo[d]imidazol-1-yl)-[1,1′-biphenyl]-3-yl)cyclopropanesulfonamide (I) as an active ingredient and an excipient which is copovidone.

### BRIEF DESCRIPTION OF THE DRAWINGS

FIG. 1 shows the X-ray powder diffraction pattern of the crystalline form 8 of hydrochloride salt of compound (I) obtained in Example 14.

FIG. 2 shows the effect of copovidone (PVP/VA) on the dissolution of compound (I) HCl salt from a tablet formulation.

FIG. 3 compares of the effect of copovidone (PVP/VA) and povidone (PVP) on the dissolution of compound (I) HCl salt from a tablet formulation.

FIG. 4 shows single dose pharmacokinetics of compound (I) HCl salt after oral dosing of suspension formulations to dogs (dose 20 mg/kg).

FIG. 5 shows single dose pharmacokinetics of compound (I) HCl salt after oral dosing of suspension formulations to dogs (dose 40 mg/kg).

FIG. 6 shows single dose pharmacokinetics of compound (I) HCl salt after oral dosing of tablet formulations to minipigs.

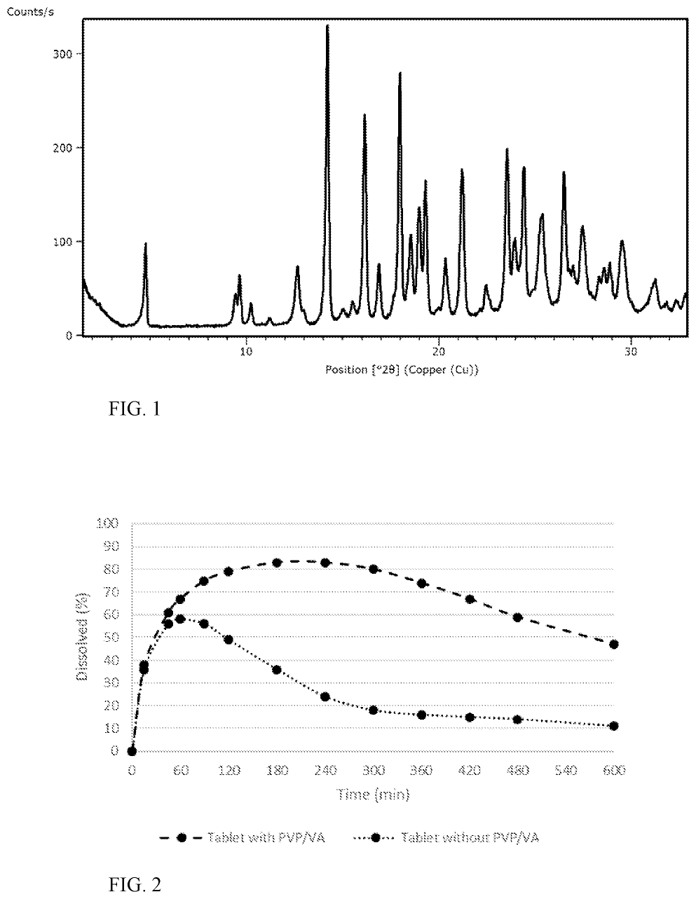

### DETAILED DESCRIPTION OF THE INVENTION

The present invention relates to pharmaceutical compositions comprising hydrochloride salt of compound (I) as an active ingredient and copovidone as an excipient. Copovidone has been found to enhance oral bioavailability of hydrochloride salt of compound (I) and is therefore particularly useful as an excipient in pharmaceutical compositions comprising hydrochloride salt of compound (I).

The term “copovidone” or “PVP/VA”, as used herein, refers to copolymer of 1-ethenylpyrrolidin-2-one and ethenylacetate. Copovidone is available e.g. under trade names Plasdone™ S-630 and Kollidon® VA64.

The term “hydrochloride salt of N-(2′,4′-difluoro-5-(5-(1-methyl-1H-pyrazol-4-yl)-1H-benzo[d]imidazol-1-yl)-[1,1′-biphenyl]-3-yl)cyclopropanesulfonamide (I)” is inclusive of amorphous, crystalline, solvated, cocrystal or solubilized form of the compound. Crystalline form is preferred. Particularly preferred is crystalline form 8 of hydrochloride salt of compound (I) having a X-ray powder diffraction pattern comprising characteristic peaks at about 4.7, 14.2, 16.1, 18.0, 21.2, 23.5 and 26.5 degrees 2-theta, more particularly at about 4.7, 9.4, 14.2, 16.1, 16.9, 18.0, 18.5, 19.0, 21.2, 23.5, 24.0, 24.4, 25.3, 26.5, 27.5 and 29.5 degrees 2-theta. In one embodiment, said crystalline form 8 is in the form of a monohydrate. XRPD measurements were performed with the X-ray powder diffractometer PANalytical X'Pert PRO at room temperature using copper filled X-ray tube (45 kV×40 mA) as the X-ray source, a fixed 1° anti-scatter slit, a programmable divergence slit with 10 mm irradiated length, and the real time multiple strip detector X'Celerator. Data collection was done in 0.017° steps at a scan speed of 0.1°/s in the range of 3-40° 2θ.

The pharmaceutical composition of the invention can be, for example, in the form of powders, granules, pellets, suspensions, capsules or tablets.

According to one embodiment of the present invention, there is provided a pharmaceutical composition comprising

(a) from about 0.1 to about 98%, preferably from about 0.2 to about 70%, more preferably from about 0.3 to about 60%, per weight of the composition, of hydrochloride salt of compound (I); and

(b) from about 0.5 to about 50%, preferably from about 1 to about 40%, more preferably from about 2 to about 35%, per weight of the composition, of copovidone.

According to one aspect of the invention, the hydrochloride salt of compound (I) is in crystalline form. According to another aspect of the invention, the hydrochloride salt of compound (I) is in crystalline form 8. According to another aspect of the invention, the crystalline form 8 is in the form of a monohydrate.

According to one aspect of the invention, the composition is in the form of a tablet.

A tablet composition according to the present invention may suitably comprise

(a) from about 10 to about 80%, preferably from about 15 to about 75%, more preferably from about 20 to about 70%, still more preferably from about 25 to about 55%, per weight of the composition, of hydrochloride salt of compound (I); and

(b) from about 1 to about 50%, preferably from about 2 to about 30%, more preferably from about 3 to about 20%, %, still more preferably from about 4 to about 15%, per weight of the composition, of copovidone.

In a subclass of any of the above embodiments are tablet compositions comprising further from about 10 to about 75%, preferably from about 15 to about 70%, more preferably from about 20 to about 65%, per weight of the composition, of a filler.

As used herein, a “filler” refers to one or more pharmaceutically acceptable excipient(s) that adds bulkiness to a pharmaceutical composition. Examples of fillers include microcrystalline cellulose, lactose, calcium hydrogen phosphate, sorbitol, starches, sugars (e.g., mannitol or sucrose) or any combination thereof. According to one preferred embodiment, the filler comprises microcrystalline cellulose.

In a subclass of any of the above embodiments are tablet compositions comprising further from about 0.5 to about 10%, preferably from about 3 to about 7%, per weight of the composition, of a disintegrant.

As used herein, a “disintegrant” refers to one or more pharmaceutically acceptable excipient(s) which is added to the pharmaceutical composition to cause its disintegration to support the release of the active ingredient from the pharmaceutical composition. Examples of disintegrants include croscarmellose sodium, cross-linked polyvinylpyrrolidone (crospovidone), sodium starch glycolate or any combination thereof. According to one preferred embodiment, the disintegrant comprises crospovidone.

In a subclass of any of the above embodiments are tablet compositions comprising further from about 0.5 to about 10%, preferably from about 3 to about 7%, per weight of the composition, of a binder.

As used herein, a “binder” refers to one or more pharmaceutically acceptable excipient(s) that imparts enhanced cohesion by binding the active ingredient and the excipients together in a mixture. Examples of binders include polyvinyl pyrrolidone (PVP), polyvinyl acetate, polyvinyl alcohol, hydroxypropylcellulose (HPC), hydroxypropylmethylcellulose (HPMC) and combinations thereof.

In a subclass of any of the above embodiments are tablet compositions comprising further from about 0.2-20%, preferably from about 1-15%, for example from about 2-12%, per weight of the composition, of a lubricant.

As used herein, a “lubricant” refers to one or more pharmaceutically acceptable excipient(s), which is added to the pharmaceutical composition to reduce friction, heat, and wear when introduced between solid surfaces. Examples of lubricants include magnesium stearate, stearic acid, talc, silica, calcium stearate, carnauba wax, sodium stearyl fumarate, and combinations thereof. According to one preferred embodiment, the lubricant comprises stearic acid.

In a subclass of any of the above embodiments are tablet compositions comprising further from about 0.5-15%, preferably from about 1-10%, for example from about 2-8%, per weight of the composition, of a glidant.

As used herein, a “glidant” refers to a material which improves the flow characteristics of powder mixtures in the dry state. Materials commonly used as a glidant include colloidal silicon dioxide or talc.

The tablet composition may also comprise other excipients known in the art such as antioxidants, colours, sweeteners, surfactants, coating agents, matrix polymers and other ingredients normally used in this field of technology may also be used.

For example, the tablet cores can be provided with a water soluble film coating, if desired, to facilitate tablet swallowing, to protect from direct contact with the drug substance and to improve aesthetics. Suitable film coating agents can be selected from the group of plasticizers, film-forming agents and colorants. Optionally an anti-tacking agent or opacifier can be used. The plasticizer, such as polyethylene glycol (PEG), the film-forming agent, such as hydroxypropylmethyl cellulose (HPMC), and the colorants, such as ferric oxide and titanium dioxide, are combined with film-coating liquids, preferably water, to result in a homogeneous coating suspension which is brought up, preferably sprayed, on the tablets in a suitable coating device, such as for example a perforated drum coater.

According to one aspect of the invention, the tablet composition comprises

(a) from about 10 to about 80%, preferably from about 15 to about 75%, more preferably from about 20 to about 70%, still more preferably from about 25 to about 55%, per weight of the composition, of hydrochloride salt of compound (I);

(b) from about 1 to about 50%, preferably from about 2 to about 30%, more preferably from about 3 to about 20%, still more preferably from about 4 to about 15%, per weight of the composition, of copovidone;

(c) from about 10 to about 75%, preferably from about 15 to about 70%, for example from about 20 to about 65%, per weight of the composition, of a filler;

(d) from about 0.2 to about 20%, preferably from about 1 to about 15%, more preferably from about 2 to about 12%, per weight of the composition, of a lubricant;

(e) from about 0.5 to about 15%, preferably from about 1 to about 10%, more preferably from about 2 to about 8%, per weight of the composition, of a glidant, and

(f) from about 0.5 to about 10%, preferably from about 3 to about 7%, per weight of the composition, of a disintegrant.

According to one aspect of the invention, the tablet composition comprises

(a) from about from about 25 to about 55%, per weight of the composition, of hydrochloride salt of compound (I);

(b) from about 4 to about 15%, per weight of the composition, of copovidone;

(c) from about 20 to about 65%, per weight of the composition, of microcrystalline cellulose;

(d) from about 2 to about 12%, per weight of the composition, of stearic acid;

(e) from about 2 to about 8%, per weight of the composition, of colloidal silicon dioxide, and

(f) from about 3 to about 7%, per weight of the composition, of crospovidone.

According to one embodiment, the tablet comprises an intragranular part and an extragranular part. According to still another embodiment, the intragranular part comprises hydrochloride salt of compound (I), copovidone, a filler, a lubricant and a glidant, and the extragranular part comprises a filler, a lubricant and the disintegrant.

According to one aspect of the invention, the tablet composition comprises
(1) an intragranular part comprising

(a) from about 25 to about 55%, per weight of the composition, of hydrochloride salt of compound (I);

(b) from about 4 to about 15%, per weight of the composition, of copovidone;

(c) from about 15 to about 45%, per weight of the composition, of a filler;

(d) from about 1 to about 12%, per weight of the composition, of a lubricant; and

(e) from about 1 to about 10%, per weight of the composition, of a glidant; and
(2) an extragranular part comprising

(f) from about 5 to about 20%, per weight of the composition, of a filler;

(g) from about 1 to about 10%, per weight of the composition, of a lubricant; and

(h) from about 0.5 to about 10%, per weight of the composition, of a disintegrant.

Tablet compositions can be prepared, for example, by dry granulation, wet granulation or direct dry compression.

Dry granulation process suitably comprises mixing the active ingredient and copovidone in a suitable blender. Other suitable ingredients such as the filler and the glidant can then be added to the mixture followed by blending. Finally, a lubricant can be added to the mixture followed by blending. The resulting mixture can then be compacted in a suitable compactor such as a roller compactor. The compacted material can then be granulated by milling in a suitable apparatus to obtain the granules for tableting. If desired, the excipients suitable for an extragranular part, such as the filler, the disintegrant and the lubricant can be mixed with the granules previously obtained (intragranular part). The resulting tablet mass can then be compressed into tablet cores in a suitable tablet press apparatus, for example, in a power assisted rotary tablet press. If desired, the obtained tablet cores can be coated with one or further pharmaceutically acceptable film-coating agents.

Wet granulation process suitably comprises mixing first the active ingredient and copovidone in a suitable blender. Other suitable ingredients such as the filler, lubricant, binder and the glidant can then be added to the mixture followed by blending. The resulting mixture is granulated using suitable granulation liquid such as water, in a suitable granulator vessel, for example wet high shear granulator. The wet granules can then be screened, for example, using a screening mill unit (rotating impeller) and subsequently dried, for example, in a fluid bed dryer. The dried granules may then be screened with a screening apparatus, for example a screening mill. If desired, the excipients suitable for an extragranular part, such as the filler, the disintegrant and the lubricant can be mixed with the granules previously obtained (intragranular part). The resulting tablet mass can then be compressed into tablet cores in a suitable tablet press apparatus, for example, in a power assisted rotary tablet press. If desired, the obtained tablet cores can be coated with one or further pharmaceutically acceptable film-coating agents.

Direct dry compression prosess comprises simply blending the active ingredient and the excipients together and compressing the dry mass into tablet cores in a suitable tablet press apparatus.

According to one embodiment of the invention the process for manufacturing a pharmaceutical composition of the invention is characterized by the steps of (a) mixing hydrochloride salt of compound (I), copovidone, filler, glidant and lubricant; (b) compacting the resulting mixture; (c) milling the compacted mixture to obtain granules; (d) mixing the resulting granules with filler, disintegrant and lubricant; (e) compressing the resulting mass into tablets; and, optionally, coating the tablet with one or further pharmaceutically acceptable film-coating agent.

According to one aspect of the invention, the composition is in form of a powder. A powder composition suitably comprises

(a) from about 25 to about 98%, preferably from about 30 to about 95%, more preferably from about 40 to about 90%, still more preferably from about 50 to about 85%, per weight of the powder, of hydrochloride salt of compound (I); and

(b) from about 1 to about 50%, preferably from about 3 to about 40%, more preferably from about 5 to about 30%, still more preferably from about 10 to about 20%, per weight of the powder, of copovidone.

In a subclass of any of the above embodiments are powder compositions comprising further from about 0.1 to about 20%, preferably from about 0.2 to about 15%, more preferably from about 0.5 to about 10%, per weight of the powder, of a lubricant.

In a subclass of any of the above embodiments are powder compositions comprising further from about 0.1 to about 20%, preferably from about 0.2 to about 15%, more preferably from about 0.5 to about 10%, per weight of the powder, of a glidant.

According to one aspect of the invention, the powder composition comprises

(a) from about 25 to about 98%, preferably from about 30 to about 95%, more preferably from about 40 to about 90%, still more preferably from about 50 to about 85%, per weight of the powder, of hydrochloride salt of compound (I); and

(b) from about 1 to about 50%, preferably from about 3 to about 40%, more preferably from about 5 to about 30%, still more preferably from about 10 to about 20%, per weight of the powder, of copovidone;

(c) from about 0.1 to about 20%, preferably from about 0.2 to about 15%, more preferably from about 0.5 to about 10%, per weight of the powder, of a lubricant; and

(d) from about 0.1 to about 20%, preferably from about 0.2 to about 15%, more preferably from about 0.5 to about 10%, per weight of the powder, of a glidant.

According to one aspect of the invention, the powder composition suitably comprises

(a) from about 40 to about 90%, per weight of the powder, of hydrochloride salt of compound (I); and

(b) from about 10 to about 20%, per weight of the powder, of copovidone;

(c) from about 0.5 to about 10%, per weight of the powder, of sodium stearyl fumarate; and

(d) from about 0.5 to about 10%, per weight of the powder, of colloidal silicon dioxide.

The powder composition can be prepared by mixing the active ingredient and the excipient in a suitable blender. If desired, the resulting mixture can be filled in a soft or hard shell capsule, for example in a gelatine or a HPMC capsule.

According to one aspect of the invention, the composition is in form of a suspension. A suspension composition suitably comprises

(a) from about 0.1 to about 20%, preferably from about 0.2 to about 10%, more preferably from about 0.3 to about 5%, per weight of the suspension, of hydrochloride salt of compound (I);

(b) from about 0.3 to about 10%, preferably from about 1 to about 8%, more preferably from about 2 to about 5%, per weight of the suspension, of copovidone; and

(c) from about 80 to about 99.5%, preferably from about 85 to about 99%, more preferably from about 90 to about 95%, per weight of the suspension, of water.

In a subclass of any of the above embodiments are suspension compositions comprising further from about 0.1 to about 10%, preferably from about 0.2 to about 5%, more preferably from about 0.3 to about 2%, per weight of the suspension, of surfactant.

According to one aspect of the invention, the suspension composition suitably comprises

(a) from about 0.1 to about 20%, preferably from about 0.2 to about 10%, more preferably from about 0.3 to about 5%, per weight of the suspension, of hydrochloride salt of compound (I);

(b) from about 0.3 to about 10%, preferably from about 1 to about 8%, more preferably from about 2 to about 5%, per weight of the suspension, of copovidone;

(c) from about 0.1 to about 10%, preferably from about 0.2 to about 5%, more preferably from about 0.3 to about 2%, per weight of the suspension, of surfactant; and

(d) from about 80 to about 99.5%, preferably from about 85 to about 99%, more preferably from about 90 to about 95%, per weight of the suspension, of water.

As used herein, a “surfactant” refers to agent that lowers the surface tension of a liquid, for example water. In general, surfactants may be selected from the group consisting of anionic surfactants, non-ionic surfactants, cationic surfactants, amphoteric surfactants, zwitterionic surfactants, and combinations thereof. Non-ionic surfactants are particularly preferred. Examples of non-ionic surfactants include fatty acid esters of sorbitol such as sorbitan monolaurate; polyoxyethylene sorbitan esters (polysorbates), such as polyoxyethylene sorbitan monooleate (polysorbate 80); poloxamers and glycerol monostearate. According to one preferred embodiment, the surfactant is polysorbate 80.

Other excipients commonly used in suspension formulations can also be added including thickening agents (for example carbomers and cellulose derivatives), pH adjusting agents, preservatives, sweeteners, flavouring agents and colouring agents.

Suspensions can be prepared by mixing the active ingredient, copovidone and the optional excipients, for example a surfactant, in water followed by stirring.

Hydrochloride salt of compound (I) is suitably administered, for example for the treatment of cancer such as multiple myeloma, gastric cancer, endometrial cancer, prostate cancer, breast cancer, cholangiocarcinoma and uroepithelial carcinoma in an amount ranging from about 50 mg to about 2000 mg, preferably from about 100 mg to about 1500 mg, more preferably from about 200 mg to about 1000 mg, for example from about 300 mg to about 800 mg, such as about 400 mg, per day to the patient. A patient is a mammal, particularly a human, in need of treatment for, for example, cancer. The dose can be administered once daily or divided to several times a day, for example twice daily. The composition of the invention, such as a tablet, may comprise hydrochloride salt of compound (I) in an amount ranging from about 50 mg to about 800 mg, preferably from about 100 mg to about 700 mg, more preferably from about 150 mg to about 600 mg, for example from about 200 mg to about 500 mg, such as 400 mg. Such composition can be administered once or several times a day, or intermittently, for example weekly or biweekly.

The invention is further illustrated by the following non-limiting examples.

### EXAMPLE 1

### Tablet Formulation

| Intragranular: | | |
|---|---|---|
| Compound (I) HCl salt | 214.4 | mg |
| Copovidone (PVP/VA) | 36.0 | mg |
| Colloidal silicon dioxide | 25.0 | mg |
| Stearic acid | 37.5 | mg |
| Microcrystalline cellulose | 99.2 | mg |
| Extragranular: | | |
| Microcrystalline cellulose | 50.0 | mg |
| Crospovidone | 25.4 | mg |
| Stearic acid | 12.5 | mg |
| TOTAL | 500.0 | mg |

The intragranular part was manufactured by mixing the active ingredient and copovidone in a blender. The microcrystalline cellulose and the colloidal silicon dioxide was then added. Finally, stearic acid was added to the mixture followed by blending. The resulting mixture was compacted. The compacted material was granulated by milling. The excipients of the extragranular part were mixed with the granules of the intragranular part. The resulting tablet mass was compressed in a tablet press apparatus.

### EXAMPLE 2

### Tablet Formulation

| Intragranular: | | |
|---|---|---|
| Compound (I) HCl salt | 214.4 | mg |
| Copovidone (PVP/VA) | 36.0 | mg |
| Colloidal silicon dioxide | 21.0 | mg |
| Magnesium stearate | 7.0 | mg |
| Microcrystalline cellulose | 299.1 | mg |
| Extragranular: | | |
| Microcrystalline cellulose | 80.5 | mg |
| Crospovidone | 35.0 | mg |
| Magnesium stearate | 7.0 | mg |
| TOTAL | 700.0 | mg |

The formulation was prepared as described in Example 1 except that stearic acid was replaced by magnesium stearate.

### EXAMPLE 3

### Tablet Formulation

| Intragranular: | | |
|---|---|---|
| Compound (I) HCl salt | 214.4 | mg |
| Copovidone (PVP/VA) | 36.0 | mg |
| Colloidal silicon dioxide | 21.0 | mg |
| Sodium stearyl fumarate | 21.0 | mg |
| Microcrystalline cellulose | 285.1 | mg |
| Extragranular: | | |
| Microcrystalline cellulose | 80.5 | mg |
| Crospovidone | 35.0 | mg |
| Sodium stearyl fumarate | 7.0 | mg |
| TOTAL | 700.0 | mg |

The formulation was prepared as described in Example 1 except that stearic acid was replaced by sodium stearyl fumarate.

### EXAMPLE 4

### Tablet Formulation

| Intragranular: | | |
|---|---|---|
| Compound (I) HCl salt | 214.4 | mg |
| Copovidone (PVP/VA) | 36.0 | mg |
| Colloidal silicon dioxide | 21.0 | mg |
| Stearic acid | 24.5 | mg |
| Crospovidone | 14.0 | mg |
| Microcrystalline cellulose | 281.6 | mg |
| Extragranular: | | |
| Microcrystalline cellulose | 80.5 | mg |
| Crospovidone | 21.0 | mg |
| Stearic acid | 7.0 | mg |
| TOTAL | 700.0 | mg |

The formulation was prepared as described in Example 1 except that crospovidone was added also in the intragranular part.

### EXAMPLE 5

### Tablet Formulation

| | | |
|---|---|---|
| Compound (I) HCl salt | 214.4 | mg |
| Copovidone (PVP/VA) | 37.6 | mg |
| Colloidal silicon dioxide | 17.9 | mg |
| Microcrystalline cellulose | 555.9 | mg |
| Crospovidone | 29.5 | mg |
| Stearic acid | 30.3 | mg |
| TOTAL | 885.6 | mg |

The formulation was prepared by mixing the active ingredient and the excipients together in a blender and compacting the powdery mixture in a tablet press into tablets.

### EXAMPLE 6

### Tablet Formulation

| Intragranular: | | |
|---|---|---|
| Compound (I) HCl salt | 214.4 | mg |
| Copovidone (PVP/VA) | 36.0 | mg |
| Colloidal silicon dioxide | 12.5 | mg |
| Stearic acid | 12.5 | mg |
| Microcrystalline cellulose | 136.7 | mg |
| Extragranular: | | |
| Microcrystalline cellulose | 50.0 | mg |
| Crospovidone | 25.4 | mg |
| Stearic acid | 12.5 | mg |
| TOTAL | 500.0 | mg |

The formulation was prepared as described in Example 1.

### EXAMPLE 7

### Reference Tablet Formulation (Without Copovidone)

| Intragranular: | | |
|---|---|---|
| Compound (I) HCl salt | 214.4 | mg |
| Colloidal silicon dioxide | 12.5 | mg |
| Stearic acid | 12.5 | mg |
| Microcrystalline cellulose | 172.7 | mg |
| Extragranular: | | |
| Microcrystalline cellulose | 50.0 | mg |
| Crospovidone | 25.4 | mg |
| Stearic acid | 12.5 | mg |
| TOTAL | 500.0 | mg |

The formulation was prepared as described in Example 1 but excluding copovidone.

### EXAMPLE 8

### Capsule Formulation

| | | |
|---|---|---|
| Compound (I) HCl salt | 107.2 | mg |
| Copovidone (PVP/VA) | 20.0 | mg |
| Colloidal silicon dioxide | 1.4 | mg |
| Sodium stearyl fumarate | 1.4 | mg |
| TOTAL | 130 | mg |

The excipients and the active ingredient were mixed and filled in hard gelatine capsule, size 0.

### EXAMPLE 9

### Suspension Formulation

| | | |
|---|---|---|
| Compound (I) HCl salt | 2.215 | g |
| Copovidone (PVP/VA) | 10 | g |
| Polysorbate 80 | 2.5 | g |
| Water | 500 | ml |

Polysorbate 80 and copovidone were mixed with water and the active ingredient was suspended in the mixture.

### EXAMPLE 10

### Effect of Copovidone (PVP/VA) on the Dissolution of Compound (I) HCl Salt from a Tablet Formulation

In vitro dissolution of compound (I) HCl salt from a tablet of Example 6 (with copovidone) and Example 7 (without copovidone) were compared. Dissolution conditions: USP Apparatus II (paddles), speed 75 rpm, medium FeSSIF pH 5.0, temperature 37° C.±0.5° C., vessel volume 500 ml, sample volume 1.3 ml (Autosampler) using 45 μm flow filters. The dissolution results are shown in FIG. 2. It can be seen that enhanced dissolution profile in FeSSIF was obtained for the tablet containing copovidone (PVP/VA). The maximum concentration of the active ingredient was higher and the active ingredient remained in solution longer than for tablet without copovidone (PVP/VA).

### EXAMPLE 11

### Comparison of the Effect of Copovidone (PVP/VA) and Povidone (PVP) on the Dissolution of Compound (I) HCl Salt from a Tablet Formulation

In vitro dissolution of compound (I) HCl salt from a tablet formulation A (containing copovidone) and B (containing povidone) were compared.

Tablet formulation A (containing copovidone):

Compound (I) HCl salt: 106.8 mg

Copovidone (PVP/VA): 17.7 mg

Sodium starch glycolate: 25.5 mg

TOTAL 150 mg

Tablet formulation B (containing povidone):

Compound (I) HCl salt: 106.8 mg

Povidone (PVP) 17.7 mg

Sodium starch glycolate: 25.5 mg

TOTAL 150 mg

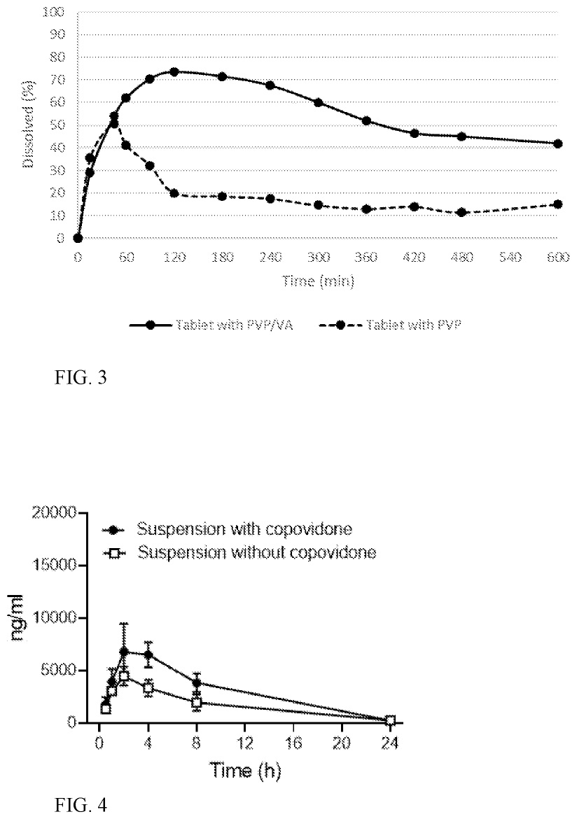

The tablet formulations were prepared by triturating the active ingredient and copovidone or povidone together. Then sodium starch glycolate was added to the mixture followed by blending in a turbular mixer. 150 mg of the mixture was weighed and compacted on a tablet press. Dissolution conditions: USP Apparatus II (paddles), speed 75 rpm, medium FeSSIF pH 5.0, temperature 37° C.±0.5° C., vessel volume 500 ml, sample volume 1.3 ml (Autosampler) using 45 μm flow filters. The dissolution results are shown in FIG. 3. It can be seen that enhanced dissolution profile in FeSSIF was obtained for the tablet containing copovidone (PVP/VA). The maximum concentration of the active ingredient was higher and the active ingredient remained in solution longer than for the tablet containing povidone (PVP).

### EXAMPLE 12

### Single Dose Pharmacokinetics of Compound (I) HCl Salt After Oral Dosing of Suspension Formulations to Dogs

A single dose of suspension formulations containing compound (I) HCl salt were administered orally to Beagle dogs (n=2) as follows.

Suspension formulation A (without copovidone):

Compound (I) HCl salt: 2.215 mg or 4.43 mg

Polysorbate 80: 2.5 g

Water: Ad 500 ml

Suspension formulation B (with copovidone):

Compound (I) HCl salt: 2.215 mg or 4.43 mg

Polysorbate 80: 2.5 g

Copovidone (PVP/VA): 10 g

Water: Ad 500 ml

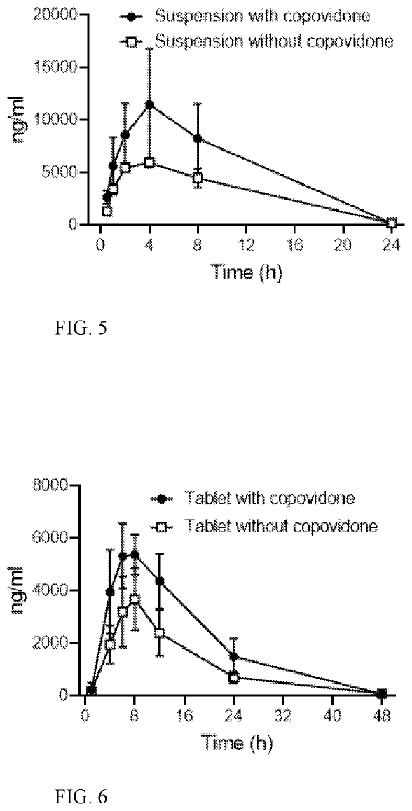

Dosing volume was 5 ml/kg and target doses 20 mg/kg and 40 mg/kg of compound (I). Blood samples were collected at different time points after the single oral dose. Plasma was separated and analysed using a LC-MS/MS method. The measured plasma concentrations (mean±SD) are shown in FIG. 4 (dose 20 mg/kg) and FIG. 5 (dose 40 mg/kg). It can be seen that the mean concentrations and exposures of the active ingredient were almost two-fold after dosing with the formulations including copovidone (PVP/VA) compared to formulations without copovidone (PVP/VA). The maximum concentrations and exposures after dosing with the formulation including copovidone (PVP/VA) were higher in all individuals (regardless of the dose) compared to the formulation without copovidone (PVP/VA).

### EXAMPLE 13

### Single Dose Pharmacokinetics of Compound (I) HCl Salt After Oral Dosing of Tablet Formulations to Minipigs

A single dose of tablet formulations of Example 6 (with copovidone) and Example 7 (without copovidone) were administered orally to male Gottingen minipigs (n=6). Blood samples were collected at different time points after the single oral dose. Plasma was separated and analysed using a LC-MS/MS method. The measured plasma concentrations (mean±SD) are shown in FIG. 6. It can be seen that the peak plasma concentration, Cmax, and systemic exposure, in terms of AUC 0-24 values, were higher after oral administration of formulation of Example 6 (with copovidone) than after oral administration of formulation of Example 7 (without copovidone).

### EXAMPLE 14

### Preparation of Hydrochloride salt of N-(2′,4′-difluoro-5-(5-(1-methyl-1H-pyrazol-4-yl)-1H-benzo[d]imidazol-1-yl)-[1,1′-biphenyl]-3-yl)cyclopropanesulfonamide (I) as Crystalline Form 8

To an inerted (N2) flask was added water (23.5 ml), 2-propanol (23.5 ml), formic acid (66 ml) and hydrochloric acid (5.21 ml, 30 w-%, 1.5 equivalents). To this solution was added N-(2′,4′-difluoro-5-(5-(1-methyl-1H-pyrazol-4-yl)-1H-benzo[d]imidazol-1-yl)-[1,1′-biphenyl]-3-yl)cyclopropanesulfonamide (18.9 g). The mixture was heated to 60±5° C. The solution was polish filtered while hot. To the filtrate was added 60 ml of 1:1 mixture of water and 2-propanol while keeping the temperature at 60±5° C. The solution was seeded, after which 70 ml more of the 1:1 water/2-propanol mixture was added while keeping the temperature at 60±5° C. The mixture was stirred for 30 min prior to allowing the mixture to cool to 20±5° C. over several hours. The mass was further cooled to 5±5° C. and stirred for 1 h prior to isolation by filtration. The cake was washed with isopropyl alcohol (50 ml) and dried in a vacuum oven at 50° C. to give 17.88 g (93.0%) of crystalline form 8 as a monohydrate.

We claim:

1. A pharmaceutical composition comprising hydrochloride salt of N-(2′,4′-difluoro-5-(5-(1-methyl-1H-pyrazol-4-yl)-1H-benzo[d]imidazol-1-yl)-[1,1′-biphenyl]-3-yl)cyclo-propanesulfonamide (I) as an active ingredient and an excipient which is copovidone.

2. A composition according to claim 1, which comprises
(a) from about 0.1 to about 98%, preferably from about 0.2 to about 70%, more preferably from about 0.3 to about 60%, per weight of the composition, of hydrochloride salt of compound (I); and
(b) from about 0.5 to about 50%, preferably from about 1 to about 40%, more preferably from about 2 to about 35%, per weight of the composition, of copovidone.

3. A composition according to claim 1 or 2, wherein the hydrochloride salt is in crystalline form.

4. A composition according to any of claims 1 to 3, which is in the form of a tablet, powder or suspension.

5. A composition according to claim 4, which is in the form of a tablet.

6. A composition according to claim 5, comprising
(a) from about 10 to about 80%, preferably from about 15 to about 75%, more preferably from about 20 to about 70%, still more preferably from about 25 to about 55%, per weight of the composition, of hydrochloride salt of compound (I); and
(b) from about 1 to about 50%, preferably from about 2 to about 30%, more preferably from about 3 to about 20%, %, still more preferably from about 4 to about 15%, per weight of the composition, of copovidone.

7. A composition according to claim 6, comprising further from about 10 to about 75%, preferably from about 15 to about 70%, more preferably from about 20 to about 65%, per weight of the composition, of a filler.

8. A composition according to claim 6 or 7, comprising further from about 0.5 to about 10%, preferably from about 3 to about 7%, per weight of the composition, of a disintegrant.

9. A composition according to any one of claims 6 to 8, comprising further from about 0.5 to about 10%, preferably from about 3 to about 7%, per weight of the composition, of a binder.

10. A composition according to any one of claims 6 to 9, comprising further from about 0.2-20%, preferably from about 1-15%, for example from about 2-12%, per weight of the composition, of a lubricant.

11. A composition according to any one of claims 6 to 10, comprising further from about 0.5-15%, preferably from about 1-10%, for example from about 2-8%, per weight of the composition, of a glidant.

12. A composition according to claim 4, which is in the form of powder.

13. A composition according to claim 12, comprising
(a) from about 25 to about 98%, preferably from about 30 to about 95%, more preferably from about 40 to about 90%, still more preferably from about 50 to about 85%, per weight of the powder, of hydrochloride salt of compound (I); and
(b) from about 1 to about 50%, preferably from about 3 to about 40%, more preferably from about 5 to about 30%, still more preferably from about 10 to about 20%, per weight of the powder, of copovidone.

14. A composition according to claim 13, comprising further
from about 0.1 to about 20%, preferably from about 0.2 to about 15%, more preferably from about 0.5 to about 10%, per weight of the powder, of a lubricant.

15. A composition according to claim 13 or 14, comprising further
from about 0.1 to about 20%, preferably from about 0.2 to about 15%, more preferably from about 0.5 to about 10%, per weight of the powder, of a glidant.

16. A composition according to claim 4, which is in the form of suspension.

17. A composition according to claim 16, comprising
(a) from about 0.1 to about 20%, preferably from about 0.2 to about 10%, more preferably from about 0.3 to about 5%, per weight of the suspension, of hydrochloride salt of compound (I);
(b) from about 0.3 to about 10%, preferably from about 1 to about 8%, more preferably from about 2 to about 5%, per weight of the suspension, of copovidone; and
(c) from about 80 to about 99.5%, preferably from about 85 to about 99%, more preferably from about 90 to about 95%, per weight of the suspension, of water.

18. A composition according to claim 17, comprising further
from about 0.1 to about 10%, preferably from about 0.2 to about 5%, more preferably from about 0.3 to about 2%, per weight of the suspension, of surfactant.
